(19) United States
(12) Patent Application Publication
(10) Pub. No.: US 2008/0207996 A1
(43) Pub. Date: Aug. 28, 2008

(54) Portable Imaging Apparatus

(75) Inventor: John Tsai, Vancouver (CA)

Correspondence Address: Marina Larson & Associates, LLC, P.O. BOX 4928, DILLON, CO, 80435, US

(73) Assignee: SOLAR INTERNATIONAL PRODUCTS, INC, Vancouver, BC (CA)

(21) Appl. No.: 11/916,323

(22) PCT Filed: Jun. 6, 2006
(86) PCT No.: PCT/CA2006/000916

§ 371 Date: Dec. 3, 2007

(30) Foreign Application Priority Data
Jun. 6, 2005 (CA) 2509590

(51) Int. Cl. A61B 1/045

(52) U.S. Cl. 600/112

(57) ABSTRACT

The present invention relates to a portable imaging apparatus for imaging an internal area of an object, such as a body cavity, organ or passage of a human or animal. The imaging apparatus comprises an endoscope and a hand holdable image-viewing device releasably connectable to a proximal end of the endoscope. The endoscope comprises an image capture optical pathway and an illumination optical pathway. The image-viewing device comprises an image detector optically couplable to the image capture optical pathway, a display in communication with the detector and a light source optically couplable to the illumination optical pathway. In use, light is transmitted from the light source to the distal end of the endoscope via the illumination optical pathway and illuminates the internal area to be viewed. An optical image of the internal area is transmitted from the distal end of the endoscope to the image-viewing device via the optical image pathway and detected by the detector. A virtually instant visual display of the image is provided on the display of the image-viewing device. A method for using the portable imaging apparatus is also disclosed.

### FIELD OF INVENTION

The present invention relates to a portable imaging apparatus for imaging an internal area of an object, such as a body cavity, organ or passage of a human or animal.

### BACKGROUND OF THE INVENTION

Images of internal areas of objects, and particularly internal body areas, are generally captured by endoscopes that are inserted into the internal area to be viewed. A typical endoscope includes an elongated flexible or rigid outer tube within which a lens system is disposed at a distal end thereof. The distal end of the endoscope is inserted into an opening in the body or other object being viewed. An image of the internal area is transmitted through the lens system in the distal end, to a proximal end of the tube for reception by a camera or an electro-optic image sensor (such as a charge coupled device or CCD). The camera generates a video signal of the optical image of the interior of the object being viewed. After undergoing suitable signal processes, the video signal may be displayed on a remote display device such as a CRT monitor or video monitor.

Presently available endoscope systems, which use remote display devices such as a CRT monitor or video monitor, have several factors that are undesirable. The most important of these undesirable factors are: (i) the bulk of the equipment that is necessary to create and display the images and their proximity to the operation site and (ii) the location and number of interconnecting elements. Traditional endoscopes require the use of a collection of electronic components commonly referred to as a video tower. This rack of equipment includes several electronic components that provide functions such as: processing of video signals from the camera, supplying power to the tower-based equipment and the camera, supplying visible light energy to the endoscope and displaying the video images to the doctor, nurse or surgeon using the equipment.

WO 2003/082075 entitled “An Integrated Visualization System” discloses a video endoscope system comprising an endoscope and a camera/light unit. The camera/light unit has an endoscope adapter for connecting the unit to an endoscope, a camera for receiving images from the optics of the endoscope, a light source and a power source. Images received by the camera are converted into video signal and transmitted to a remote receiver where they can be view by the user. The system disclosed therefore has a remote image-viewing device for viewing the image, which increases the number of components required to enable a user to view the image on site.

A portable imaging apparatus, which allows a user to view the image on site without the requirement to transfer the images to a remote image-viewing device, is required.

### SUMMARY OF THE INVENTION

The present invention relates to a portable imaging apparatus for imaging an internal area of an object, such as an internal body cavity, organ or passage of a human or animal.

The portable imaging apparatus of the present invention comprises:
an endoscope comprising an image capture optical pathway and an illumination optical pathway; and
a hand holdable image viewing device releasably connectable to a proximal end of the endoscope, the device comprising:
   (i) an image detector optically couplable to the image capture optical pathway, for detecting an image transmitted from a distal end of the endoscope;
   (ii) a display in communication with the detector, for displaying an image detected by the detector; and
   (iii) a light source optically couplable to the illumination optical pathway, for transmitting light to the distal end of the endoscope.

The image-viewing device of the imaging apparatus of the present invention preferably includes a power source, which is preferably rechargeable. The light source of the image-viewing device preferably comprises a light booster system comprising a Light Emitting Diode (LED) in communication with a lens and a programmable chip.

In a preferred aspect of the imaging apparatus of the present invention, the image-viewing device comprises a mobile phone or a Personal Digital Assistant (PDA).

In the endoscope of the imaging apparatus of the present invention, the image capture optical pathway preferably comprises a plurality of image capture fibre optics and the illumination optical pathway preferably comprises a plurality of illumination fibre optics. The illumination fibre optics preferably surrounds the image capture fibre optics along at least a distal portion of the endoscope. The proximal end of the endoscope preferably comprises an adaptor realisably connectable to the image-viewing device. The adaptor preferably comprises a first connector in optical communication with the illumination optical pathway and a second connector in optical communication with the image capture optical pathway.

The image-viewing device of the apparatus of the present invention preferably includes a port releasably connectable to the proximal end of the endoscope. The port preferably comprises an image connector optically couplable with the image capture pathway of the endoscope and an illumination connector optically couplable with the illumination pathway of the endoscope.

The portable imaging apparatus of the present invention preferably includes a removable sheath configured to cover a distal portion of the endoscope, the sheath comprising an endoscope conduit with a closed distal end and an open proximal end for receiving the distal end of the endoscope. The sheath preferably comprises a substantially liquid impermeable material, preferably a substantially gas and liquid impermeable material, preferably a substantially transparent latex material.

In one aspect of the present invention, the sheath of the portable imaging apparatus includes an inflatable balloon approximate the distal end of the sheath. The balloon is preferably connected to an inflator by an inflation conduit, wherein the balloon is inflated by fluid passing from the inflator to the balloon via the inflation conduit. The inflator preferably comprises a syringe connected to the inflation conduit.

In another aspect of the present invention, the sheath of the portable imaging apparatus includes a suction conduit with an open distal end at or adjacent the distal end of the sheath and a proximal end connected to suction means. The suction means preferably comprises a syringe.

In a further aspect of the present invention, the sheath of the portable imaging apparatus includes a grasper at the distal end thereof for grasping an object.

The present invention also provides a hand holdable image viewing device comprising:
(i) a port releasably connectable to a proximal end of an endoscope, the port comprising an image connector optically couplable with an image capture pathway of the endoscope and an illumination connector optically couplable with an illumination pathway of the endoscope;
(ii) an image detector optically coupled to the image connector;
(iii) a display in communication with the detector; and
(iv) a light source optically coupled to the illumination connector.

The image-viewing device of the present invention preferably includes a power source, which is preferably rechargeable. The light source preferably comprises a light booster system comprising a Light Emitting Diode (LED) in communication with a lens and a programmable chip.

In a preferred aspect of the present invention the image viewing device comprises a mobile phone or a Personal Digital Assistant (PDA).

The present invention further provides use of the portable imaging apparatus of the present invention for imaging an internal area of an object, wherein the distal end of the endoscope is inserted into the internal area to be imaged, light is transmitted from the image viewing device to the distal end of the endoscope to illuminate the internal area and an image of the internal area is transmitted from the distal end of the endoscope to the image viewing device, whereby the image is detected by the detector and displayed on the display.

The present invention further provides use of the portable imaging apparatus of the present invention for imaging an internal body area of a human or animal, wherein the distal end of the endoscope is inserted into the internal body area to be imaged, light is transmitted from the image viewing device to the distal end of the endoscope to illuminate the internal body area and an image of the internal body area is transmitted from the distal end of the endoscope to the image viewing device, whereby the image is detected by the detector and displayed on the display.

The present invention further provides a method of viewing an image of an internal area of an object using the portable imaging apparatus of the present invention. The method comprising inserting the endoscope into the internal area to be imaged, transmitting light from the image viewing device to the distal end of the endoscope to illuminate the internal area and transmitting an image of the internal area from the distal end of the endoscope to the image viewing device, whereby the image is detected by the detector and displayed on the display.

The present invention further provides a method of viewing an image of an internal body area of a human or animal using the portable imaging apparatus of the present invention. The method comprising inserting the endoscope into the internal body area, transmitting light from the image viewing device to the distal end of the endoscope to illuminate the internal body area and transmitting an image of the internal body area from the distal end of the endoscope to the image viewing device, whereby the image is detected by the detector and displayed on the display.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a perspective view of an example of the portable imaging apparatus of the present invention comprising a hand-held image-viewing device connected to an endoscope.

FIG. 2 shows an example of the image-viewing device of FIG. 1. FIG. 2A shows a perspective view of the device, FIG. 2B shows a front view of the device, and FIG. 2C shows a back view of the device.

FIG. 3 shows an example of a light boosting system provided in the image-viewing device of FIG. 2. FIG. 3A shows a side view of the light boosting system, and FIG. 3B shows a front view of the light boosting system.

FIG. 4 shows a perspective view of the head of the light boosting system of FIG. 3.

FIG. 5 shows a perspective view of the parts that make up the head of the light boosting system as shown in FIG. 4.

FIG. 6 shows a perspective view of the portable imaging apparatus of FIG. 1 showing an adaptor for connecting the endoscope to the image-viewing device.

FIG. 7 shows the adaptor of FIG. 6. FIG. 7A shows a perspective view of the adaptor and FIG. 7B shows a side view of the adaptor connected to the image-viewing device.

FIG. 8 shows a back view of the adaptor of FIG. 7.

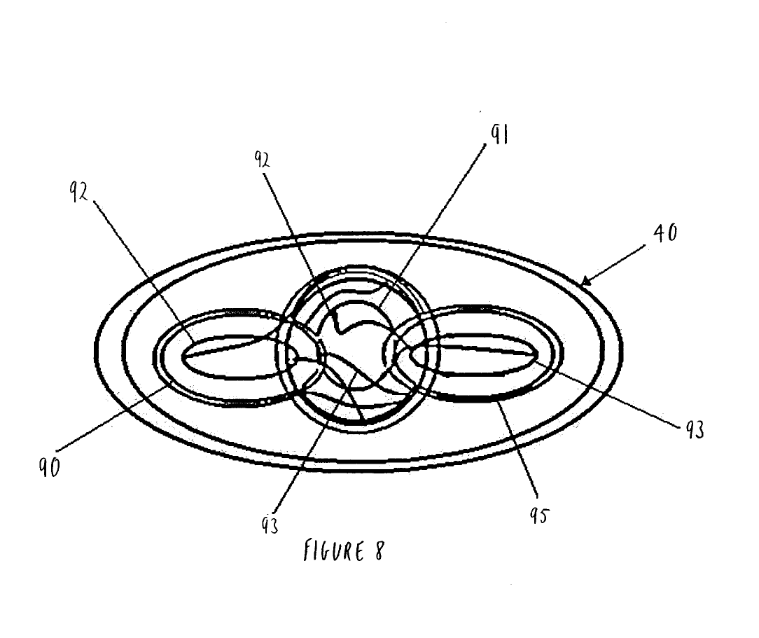

FIG. 9 shows an enlarged side view of the endoscope of FIG. 6.

FIG. 10 shows an enlarged top view of the endoscope of FIG. 6.

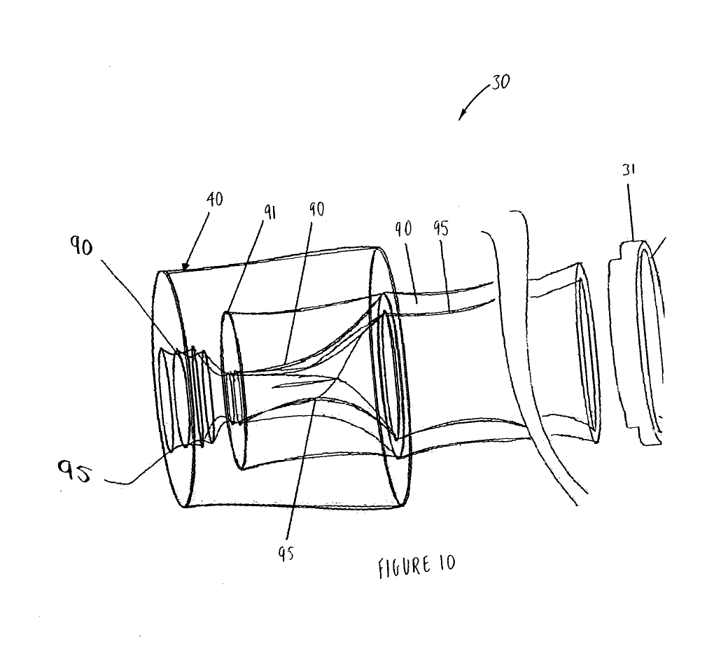

FIG. 11 shows a virtual 3D view of the imaging potential of the imaging apparatus of the present invention.

FIG. 12 shows a further virtual 3D view of the imaging potential of the imaging apparatus of the present invention.

FIG. 13 shows a comparison of two photo images captured by the image-viewing device of the present invention. FIG. 13A shows the photo image displayed by a device without a light boosting system, and FIG. 13B shows the photo image displayed by a device with a light boosting system.

FIG. 14 shows a further example of the apparatus of the present invention wherein the endoscope includes a removable balloon sheath on a distal portion of the endoscope.

FIG. 15 shows an enlarged side view of the balloon of the sheath shown in FIG. 14.

FIG. 16 shows an enlarged top view of the balloon of the sheath shown in FIG. 14.

FIG. 17 shows a perspective view of the endoscope shown in FIG. 14 with an enlarged view of the junction connecting an inflator for inflating and deflating the balloon.

FIG. 18 shows a perspective view of the balloon sheath shown in FIG. 14 into which the distal end of the endoscope is inserted.

FIG. 19 shows a further example of the apparatus of the present invention wherein the endoscope includes a removable suction sheath on a distal portion of the endoscope.

FIG. 20 shows a perspective view of the suction sheath shown in FIG. 19 into which the distal end of endoscope is inserted.

FIG. 21 shows examples of use of the apparatus shown in FIG. 14. FIG. 21A shows a front view, and FIG. 21B shows a side view of the apparatus in use for imaging the male urinary system. FIG. 21C shows a front view, and FIG. 22D shows a side view of the apparatus in use for imaging the female urinary system.

FIG. 22 shows an example of use of the apparatus shown in FIG. 14. FIG. 22A shows the positioning of the apparatus to obtain visual images of the female reproductive system. FIG. 22B shows a schematic view of the female reproductive system and FIG. 22C shows an enlarged view of the endoscope with the balloon inflated inside the uterus.

FIG. 23 shows an enlarged cross-sectional view of the distal end of the endoscope of the apparatus of the present invention indicating the flow of light and flow of optical images through the endoscope.

FIG. 24 shows a further example of the apparatus of the present invention wherein the endoscope includes a removable sheath on a distal portion of the endoscope, the sheath including a grasper for grasping an object in the internal area being viewed. FIG. 24A shows a perspective view of the sheath and FIG. 24B shows an enlarged view of the grasper.

FIG. 25 shows a perspective view of an example of a sheath for covering the distal end of the endoscope of the imaging apparatus of the present invention.

FIG. 26 shows a side view of a further example of the endoscope of the imaging apparatus of the present invention.

FIG. 27 shows a further example of the adaptor of FIG. 6. FIG. 27A shows an end view of the adaptor and FIG. 27B shows a side view of the adaptor connected to the image-viewing device.

### DETAILED DESCRIPTION OF PREFERRED EMBODIMENTS

The present invention relates to a portable imaging apparatus for imaging an internal area, for example an internal body cavity, organ or passage of a human or other animal.

The portable imaging apparatus of the present invention comprises:
an endoscope comprising an image capture optical pathway and an illumination optical pathway; and
a hand holdable image viewing device releasably connectable to a proximal end of the endoscope, the device comprising:
   (i) an image detector optically couplable to the image capture optical pathway, for detecting an image transmitted from a distal end of the endoscope;
   (ii) a display in communication with the detector, for displaying an image detected by the detector; and
   (iii) a light source optically couplable to the illumination optical pathway, for transmitting light to the distal end of the endoscope.

By the term “endoscope” it is meant a flexible or rigid viewing instrument with capabilities of diagnostic or even therapeutic function. This type of viewing instrument may sometimes be referred to as a “catheter” which also falls within the scope of the present invention, provided it comprises an image capture optical pathway and an illumination optical pathway.

The imaging apparatus of the present invention is portable and allows a user to visualize an optical image of an internal area of an object that is displayed on the display of the image-viewing device. There is therefore no need to transfer the image to an external viewer such as a TV or video monitor or a computer for the image to be viewed. A quick, virtually instantaneous visual image of an internal area of an object can be obtained from the portable imaging apparatus of the present invention. As the image-viewing device is small enough to be held in the hand, the device is easy to use, store and carry. The portable imaging apparatus of the present invention is particularly suitable for use by health personnel, such as doctors and nurses, to easily and quickly obtain a visual image of an internal body area particularly for diagnostics.

In a preferred embodiment of the present invention, the image-viewing device is a mobile phone that has been adapted be releasably connectable to an endoscope.

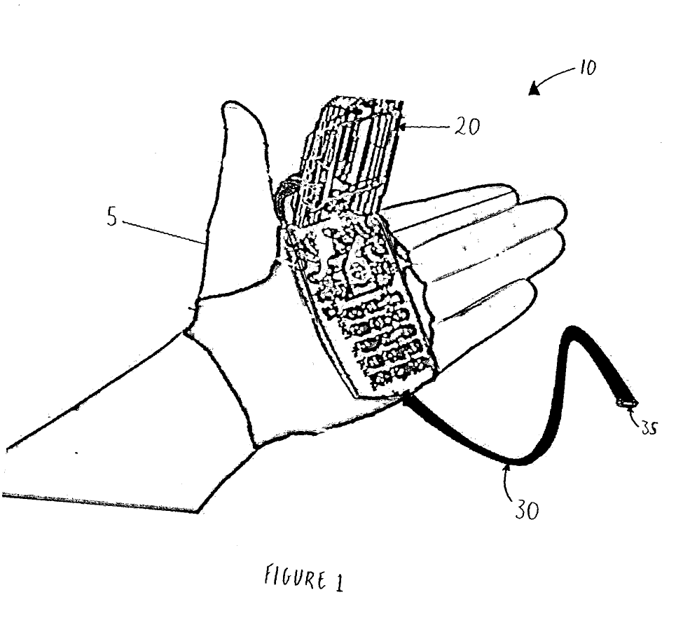

Referring to the drawings and initially to FIG. 1, there is shown an example of a portable imaging apparatus (10) of the present invention comprising a mobile phone (20) releasably connected to an endoscope (30). The distal end of the endoscope (35) can be inserted into a body cavity or body organ, via a body channel or passages such as the urethra. Light is emitted from the distal end of the endoscope (35) to illuminate the internal body cavity and the image of the cavity is transmitted along the endoscope (30) to the image-viewing device (20), allowing health personnel, such as a doctor or nurse, to instantly view the image.

In the Figures, the image-viewing device is an adapted mobile phone (20), which can be easily held in the palm of a human hand (5). It is within the scope of the invention, however, that other image viewing devices can be used in the apparatus of the present invention, for example an adapted handheld Personal Digital Assistant (PDA), an adapted digital camera, or the like. In a preferred embodiment of the invention, the image-viewing device comprises a Personal Digital Assistant (PDA).

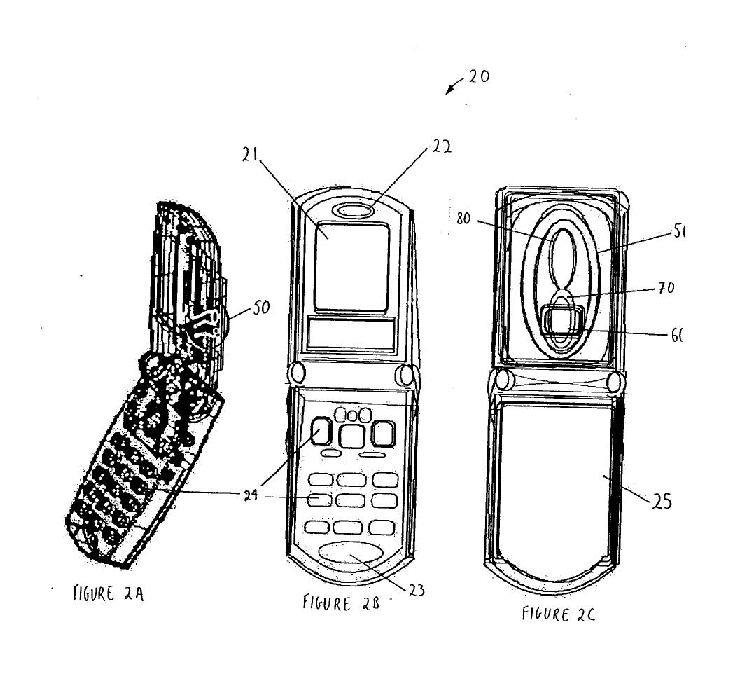

As shown in FIG. 2, the mobile phone (20) comprises a Liquid Crystal Display (LCD) screen (21) for displaying an image; an ear phone (22) and voice transmitter (23), so that the mobile phone can still be used as a means of communication; programmable software capable of manipulating and storing an image; control buttons (24); a battery (25); and an Universal Serial Bus (USB) port (not shown) to enable the phone to be connected to a computer for transfer of a stored image or images. As with standard mobile phones, the battery (25) may be rechargeable allowing the device (20) to work for a period of time before it needs to be recharged (typically 8 hours of constant communication time and up to 140 hours of stand by period).

The mobile phone (20) may be provided with other accessories, such as attachment means for attaching the phone to a belt or wrist or the like, headphones, a separate microphone and the like.

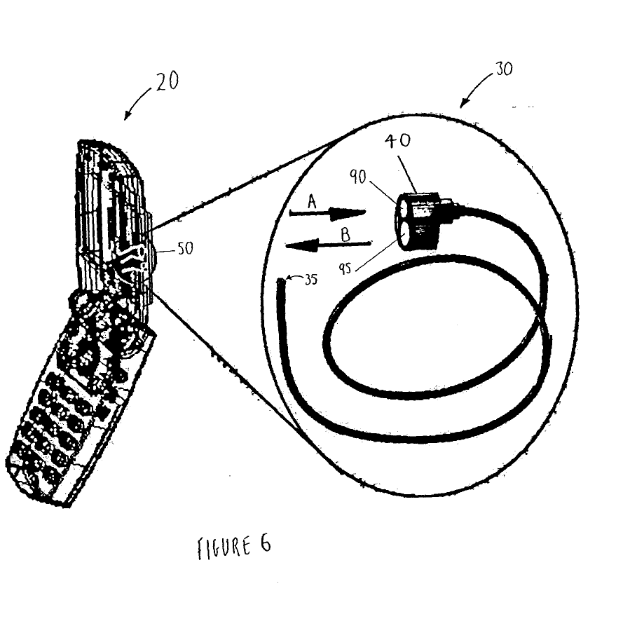

The back of the mobile phone (20) has a port (50) to releasably receive an adaptor (40) of the endoscope (30) as shown in FIG. 6. The port (50) has a substantially oval shape and includes a Charge-Coupled Device (CCD) image sensor (60) linked to an optical focusing lens (70) and a light booster system connector (80).

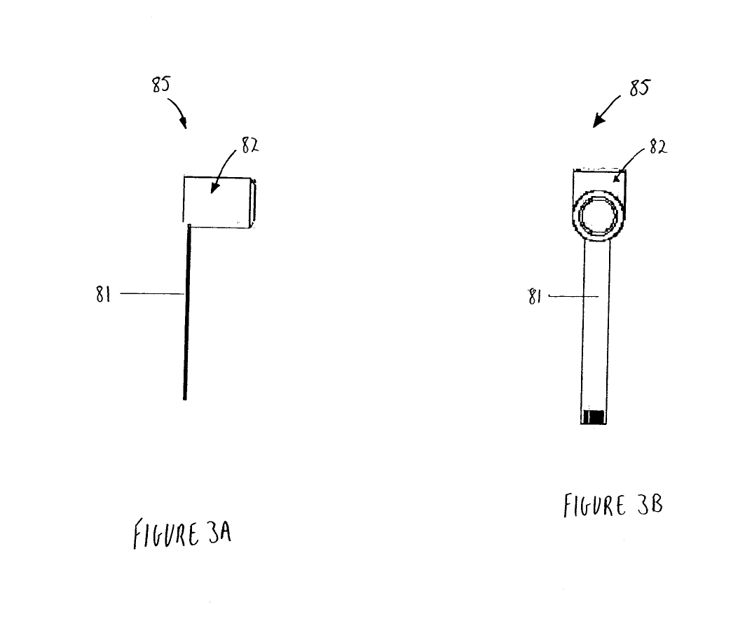

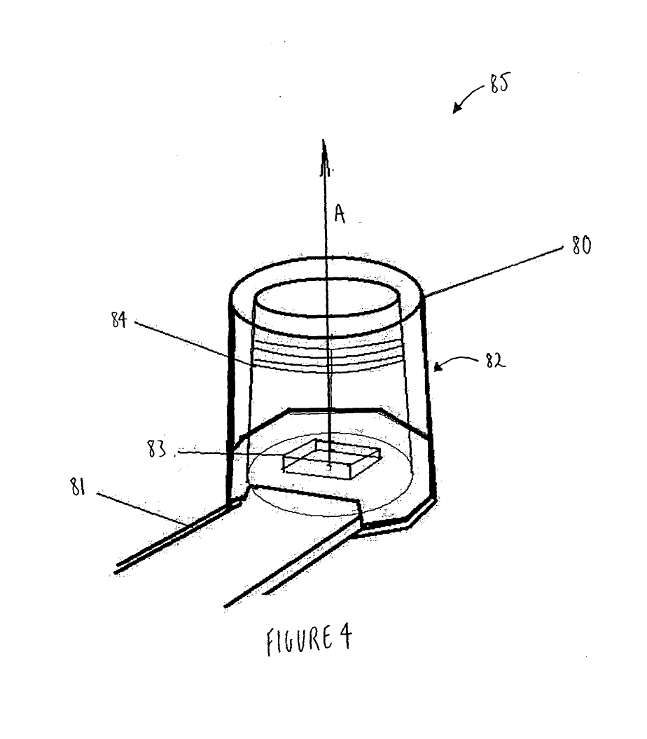

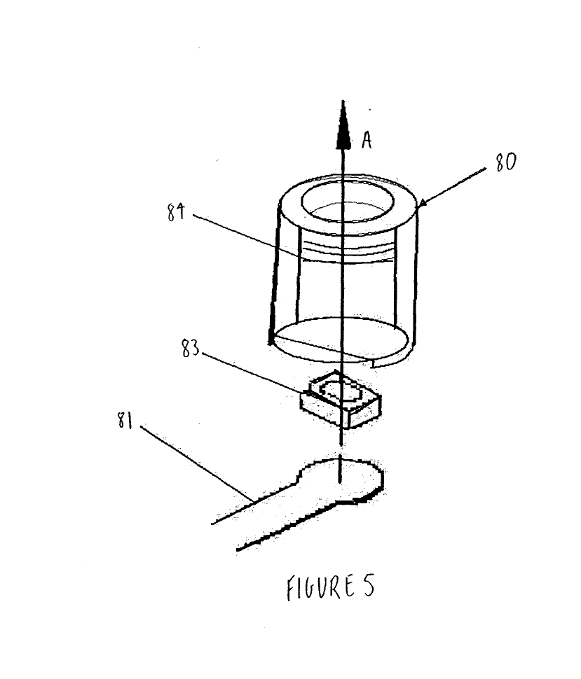

A light booster system (85) is incorporated in the body of the mobile phone (20) and comprises a cylindrical head (82) and an elongate body (81) as shown in FIGS. 3 to 5. The booster system (85) is typically about 6 mm in height and about 2 mm in width. The elongate body (81) comprises a FPC programmable chip. The head (82) comprises a Light Emitting Diode (LED) (83) positioned on the FPC chip (81) and a micro optical focus lens (84), which focuses the light emitted by the LED in Direction A (see FIGS. 4 and 5). The micro optical focus lens (84) may be made using plastic injection moulding. The upper surface of the head lies flush with the body of the mobile phone (20) and provides the light booster system connector (80) of the port (50). The FPC chip can be pre-programmed and allows a user to adjust the brightness of light being emitted from the light booster system (85) and thus from the distal end (35) of the endoscope (30). The user can make brightness adjustments by depressing a button (24) on the phone (20).

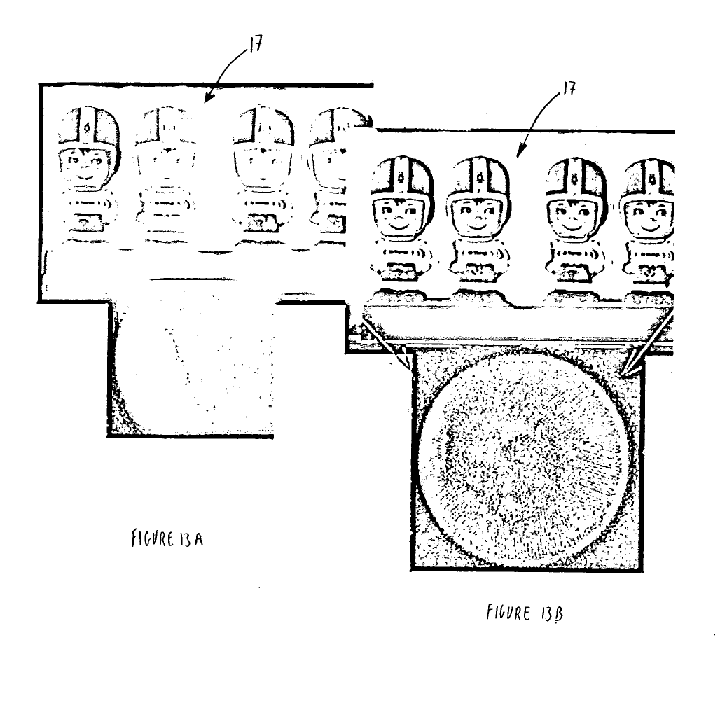

FIG. 13 shows a comparison of optical images displayed by a portable imaging apparatus (10) of the present invention having an image viewing device (20) with and without the light booster system (85). An image (17) displayed by the image viewing device (20) with the light booster system (85) is shown in FIG. 13B, and is considerably more detailed and clearer than an image displayed by the image viewing device (20) with no light booster system (85) as shown in FIG. 13B. The light booster system (85) therefore advantageously provides more focused light for illuminating the image to be viewed, which results in a clearer image displayed by the image-viewing device (20). Only one NICHIA NSCW 455 white constant flash LED is required to provide enough light to illuminate the internal spaces of the body using the apparatus of the present invention. The LED (83) is typically powered by battery (25).

A user is able to instantly view visual images (17) captured by the endoscope (30) and displayed on the LCD screen (21). The mobile phone (20) may have video camera functions as well as a still photo function, for providing a continuous video image of the internal cavity or organ. Additionally, the mobile phone (20) is able to store the captured images, along the lines of a standard mobile picture phone, and the images can be later transferred to another communication device or a computer typically through an USB port on the phone, however, other ways of transferring electronic data, for example wireless or infrared transfer, are well known in the art. The phone (20) can also be plugged into a TV monitor, to enable a larger image to be displayed on the TV monitor.

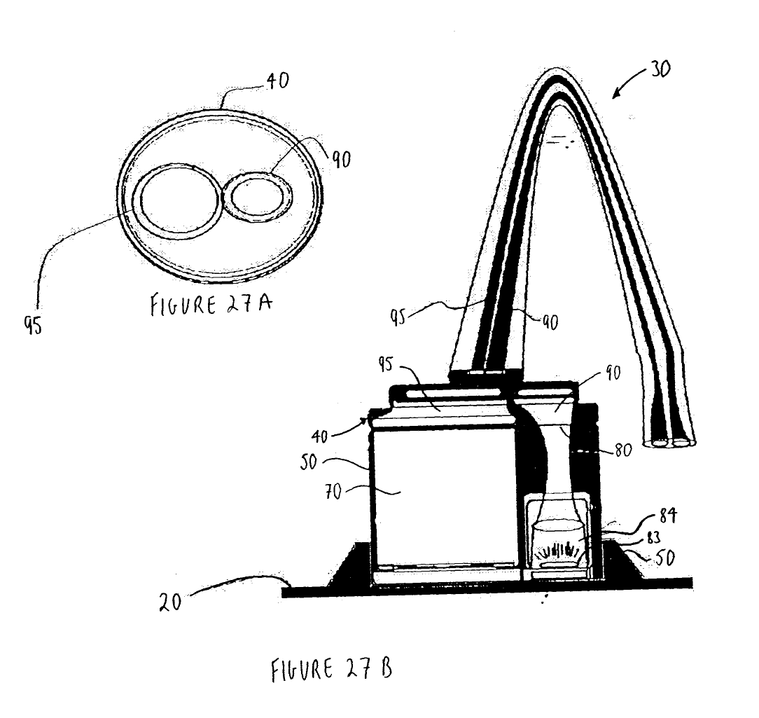

As shown in FIGS. 6, 7B, and 27 the adaptor (40) for the endoscope (30) releasably connects with the port (50) of the mobile phone (20). When the endoscope adaptor (40) is connected to port (50), the light booster system connector (80) mates with a light source conduit (90) that transports light in Direction A to the distal end (35) of the endoscope. Additionally, the optical focusing lens (70) mates with an optical image conduit (95) that transmits an image from the distal end (35) of the endoscope to the CCD image sensor (60) in Direction B. In the adapter shown in FIG. 27, the optical image conduit (95) is about twice the size of the light source conduit (90) at the point of connection with the port (50). The optical focusing lens (70) is correspondingly about twice the size of the light booster system connector (80).

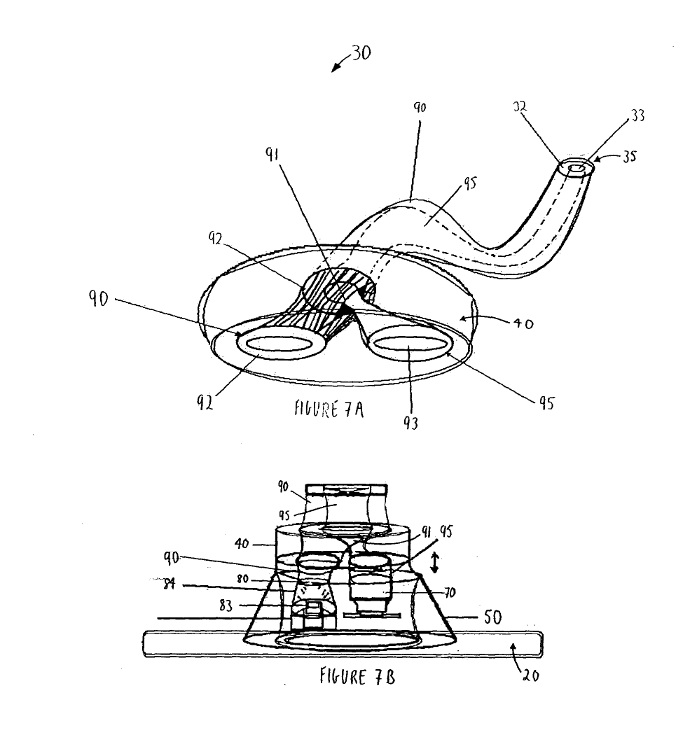

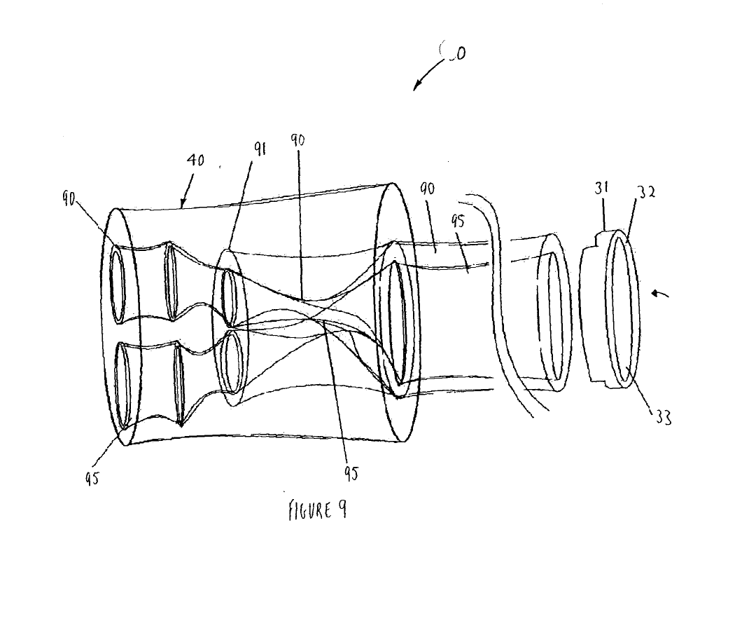

As shown in FIG. 7, the light source conduit (90) comprises a bundle of optical fibres (92) surrounded by an outer casing and the optical image conduit (95) comprises a bundle of optical fibres (93) surrounded by an outer casing. There may be more than 7200 optical fibres in each bundle of fibres (92, 93). In the embodiment shown in FIG. 7, the light source conduit (90) and the optical image conduit (95) join at junction (91) and the light source conduit (90) surrounds the optical image conduit (95) for the remaining length of the endoscope to the distal end (35). At the distal end (35) there is positioned a micro optical focus lens (31) as shown in FIG. 9, which comprises an inner optical focus lens (33) and an outer optical focus lens (32). The inner optical focus lens (33) focuses optical images of the internal area. The focused images are transmitted along optical fibres (93) to the CCD image sensor (60) in the mobile phone (20). The outer optical focus lens (32) focuses and emits light generated by the light booster system (85), which light has traveled along optical fibres (92) to reach lens (32) at the distal end (35) of the endoscope (30).

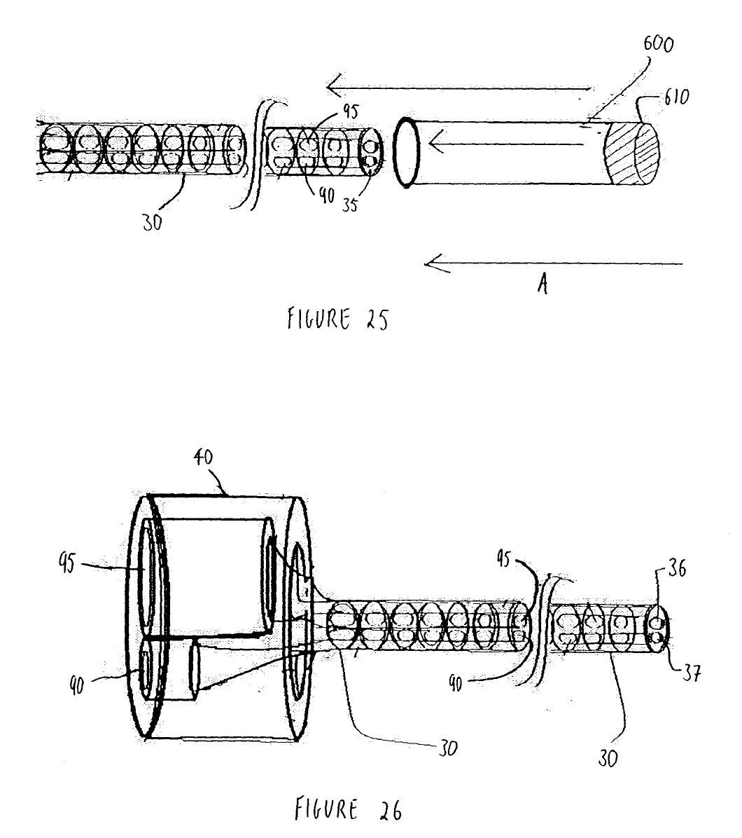

In an alternative embodiment shown in FIG. 26, the light source conduit (90) is separate from the optical image conduit (95) along the entire length of the endoscope (30). At the distal end of the optical image conduit (95) is positioned an optical image focus lens (36) for focusing optical images of the internal area. A light focus lens (37) is positioned at the distal end of the light source conduit (90) for focusing light generated by the light booster system (85), which light has traveled along optical fibres (92) to reach lens (32) at the distal end (35) of the endoscope (30).

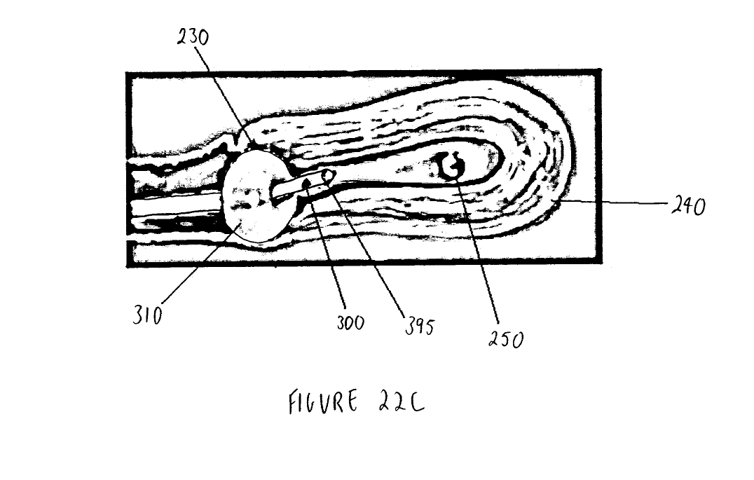

Light emitted by the outer focus lens (32) or the light focus lens (37) illuminates the internal spaces of the body cavity or passage. The image is focused by the inner optical focus lens (33) or the optical image focus lens (36) and transmitted along optical fibres (93) to the CCD image sensor (60) of the mobile phone (20). As shown in FIG. 23, light from the light booster system (80) travels along optical fibres (92) by bouncing off the casing surrounding the fibres (92), based on the principle of Total Internal Reflection. The light is then focused by the outer optical focus lens (32) or the light focus lens (36) and emitted into the internal body space or cavity. Similarly, an optical image is transmitted along optical fibres (93) by Total Internal Reflection as the image bounces off the casing surrounding the fibres (93).

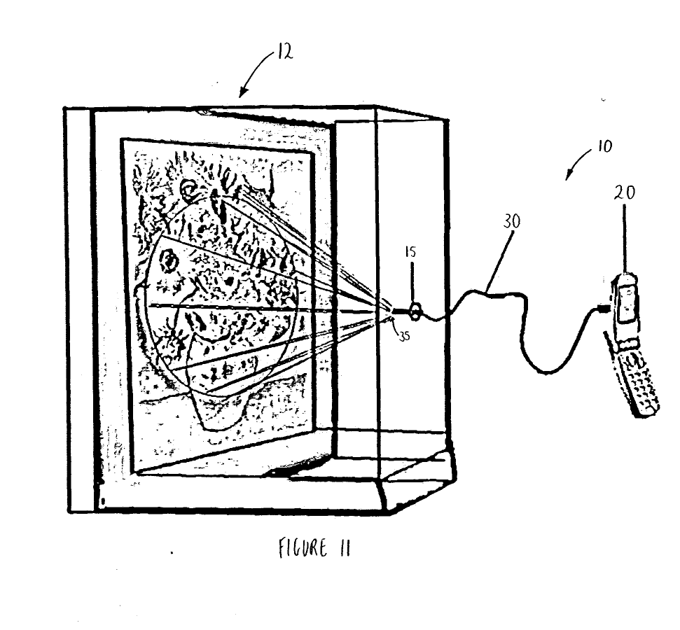

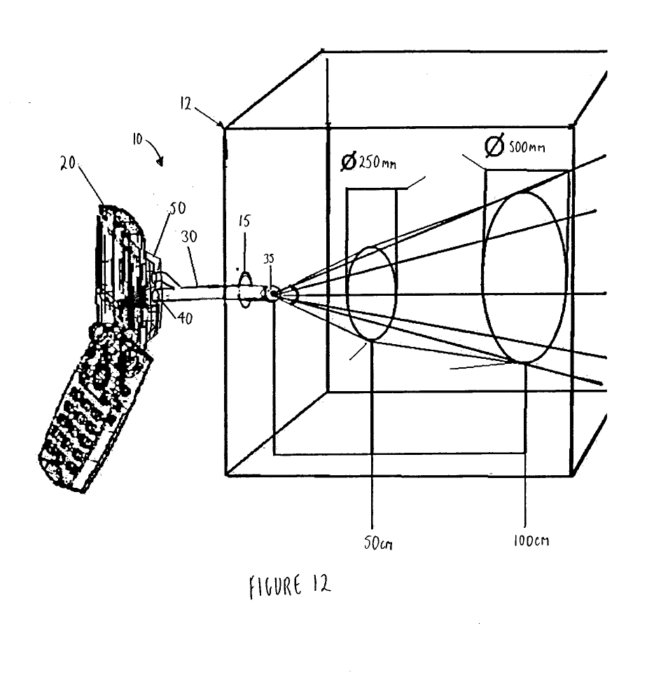

FIGS. 11 and 12 show virtual 3D views of the image capture ability of the portable imaging apparatus (10) of the present invention when the distal end (35) of the endoscope (30) is inserted through a small hole (15) of a virtual body cavity (12). As shown in FIG. 12, the light intensity at 50 cm and 100 cm from the distal end (35) of the endoscope (30) was measured and was found to cover a diameter of up to 250 mm and 500 mm respectively.

A preferred embodiment of the portable imaging apparatus of the present invention includes a sheath configured to removable cover a distal portion of the endoscope, the sheath comprising an endoscope conduit with a closed distal end and an open proximal end for receiving the distal end of the endoscope. The sheath preferably comprises a substantially liquid impermeable material, preferably a substantially gas and liquid impermeable material, preferably a substantially transparent latex material.

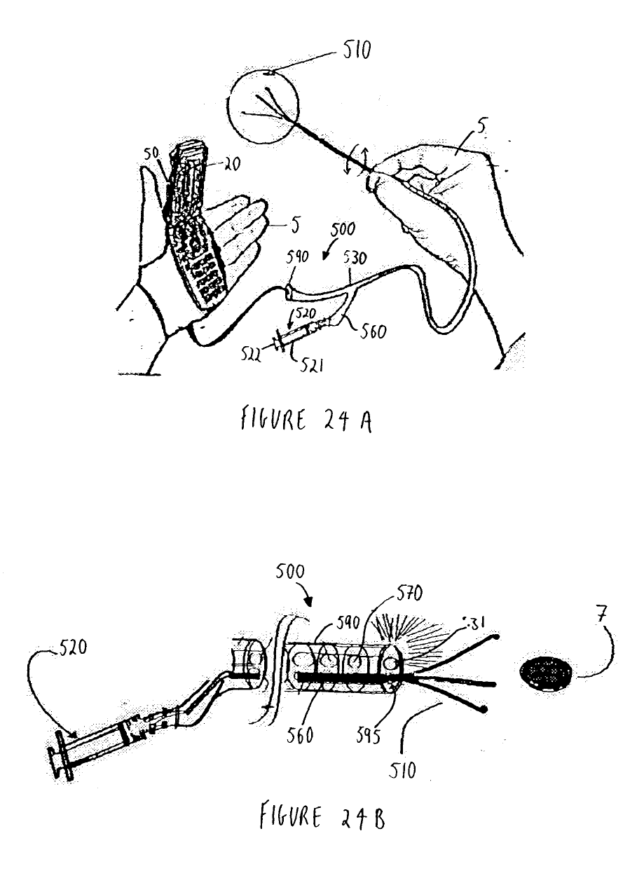

FIG. 25 shows an example of a sheath (600) that can be placed on the distal end (35) of an endoscope (30). The sheath (600) is typically sterile and may be disposable, so that the sheath (600) can be disposed after use and replaced with a further sterile sheath before the next use. This means that it is not necessary to sterilise the endoscope in between each application, instead a sterile sheath is provided to cover at least the distal end of the endoscope, which is inserted into the body cavity or passage. The sheath (600) is preferably made of a liquid and gas impermeable material, preferably a clear latex material and may come in many different lengths and sizes for use with different sized endoscopes and for different procedures. The body of the sheath (600) can be of a non-transparent material provided the distal end (610) of the sheath (600) is of a transparent material to allow an image to be picked up by the optical image conduit (95) of the endoscope (30).

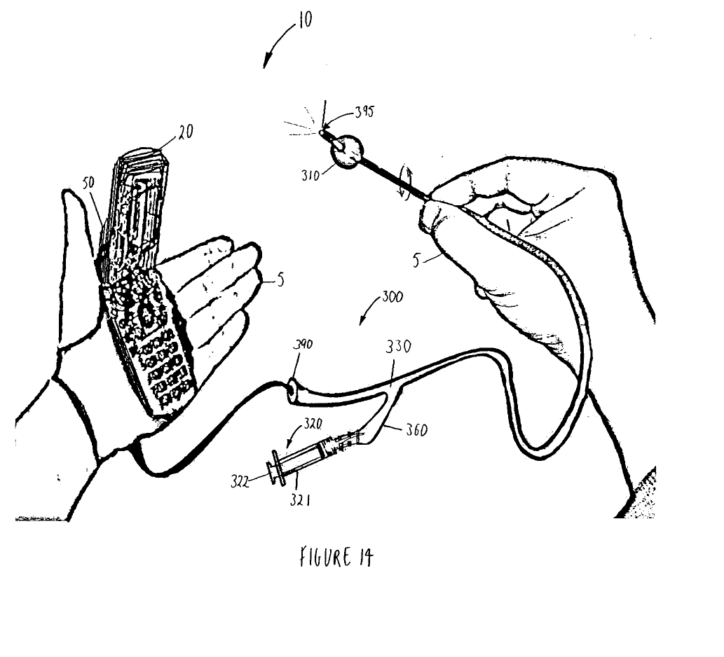

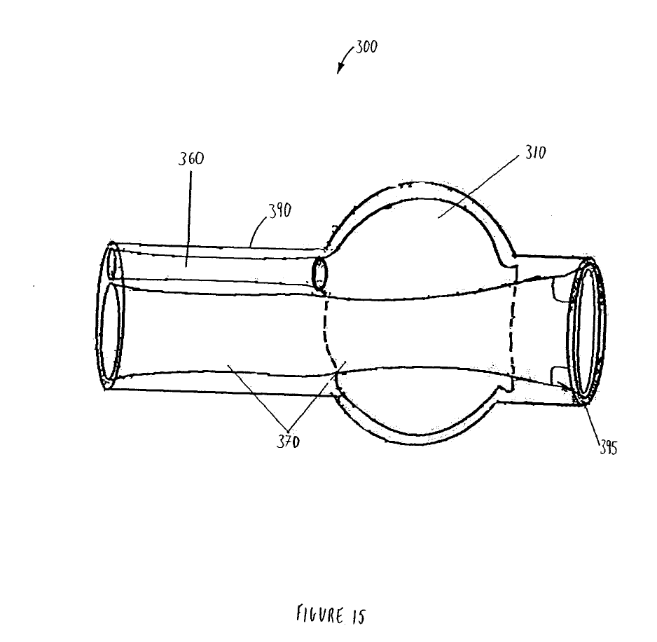

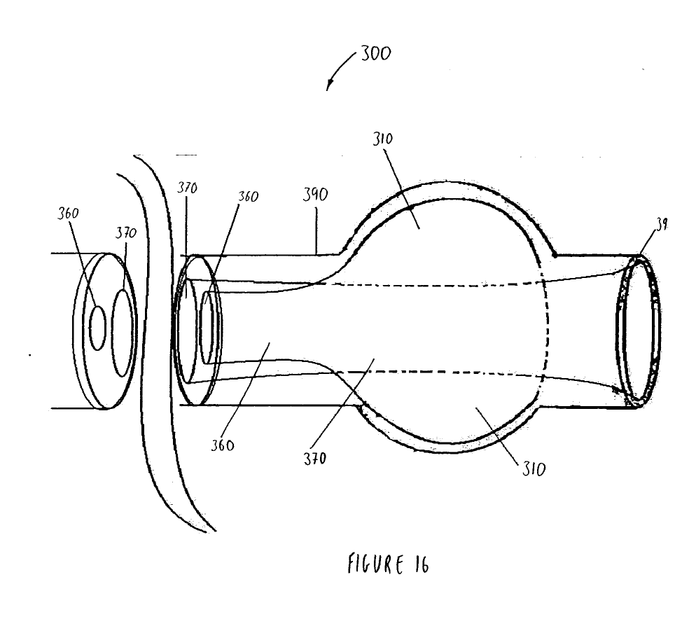

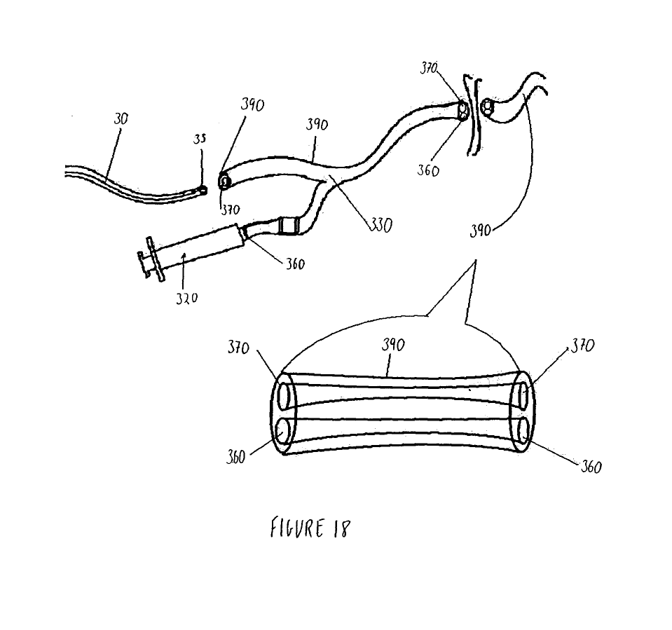

In a further preferred embodiment of the present invention, the sheath of the portable imaging apparatus includes an inflatable balloon approximate the distal end of the sheath. FIGS. 14 to 18 show an example of the portable imaging apparatus of the present invention with the endoscope (300) having a balloon sheath (390) comprising an inflatable balloon (310) connected by an inflation tube (360) to a syringe (320). The syringe (320) has a body (321) holding a fluid such as air or a liquid, and a plunger (322). The end of the body (321) of the syringe (320) is connected to inflation tube (360) so that when the plunger (322) is pushed in direction A, fluid from within body (321) passes into inflation tube (360) and inflates balloon (310). To deflate balloon (310) the plunger is pulled out in the opposite direction and the fluid in the balloon (310) is sucked back into the body (321) of the syringe (320).

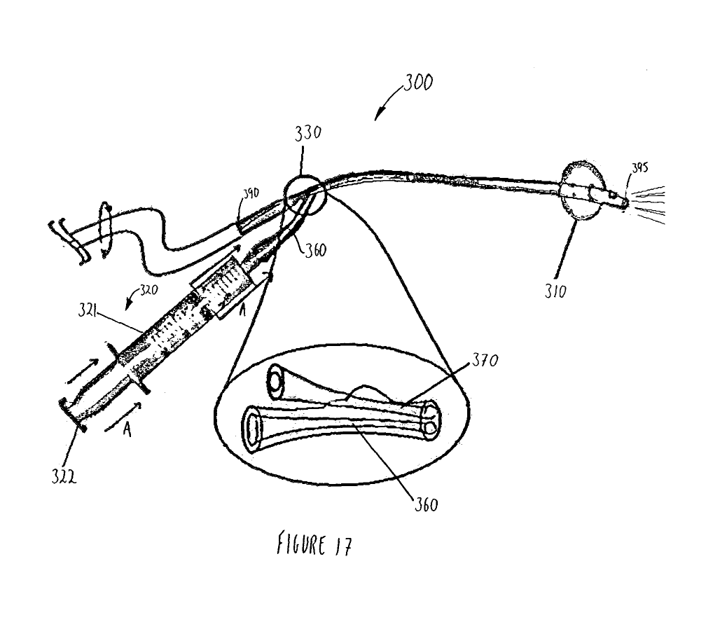

Balloon sheath (390) has an endoscope tube (370) for receiving the endoscope (30). The distal end (35) of the endoscope is inserted into endoscope tube (370) until it reaches the closed distal end of the sheath (395), so that the sheath (390) surrounds the distal portion of the endoscope (30). At junction (330) of the sheath (390), inflation tube (360) meets endoscope tube (370), which in use contains endoscope (30) as shown in FIG. 17. The sheath (390) is preferably substantially liquid and gas impermeable and acts as a barrier to protect the endoscope (30) and in particular the lens (31) at the distal end (35) of the endoscope (30).

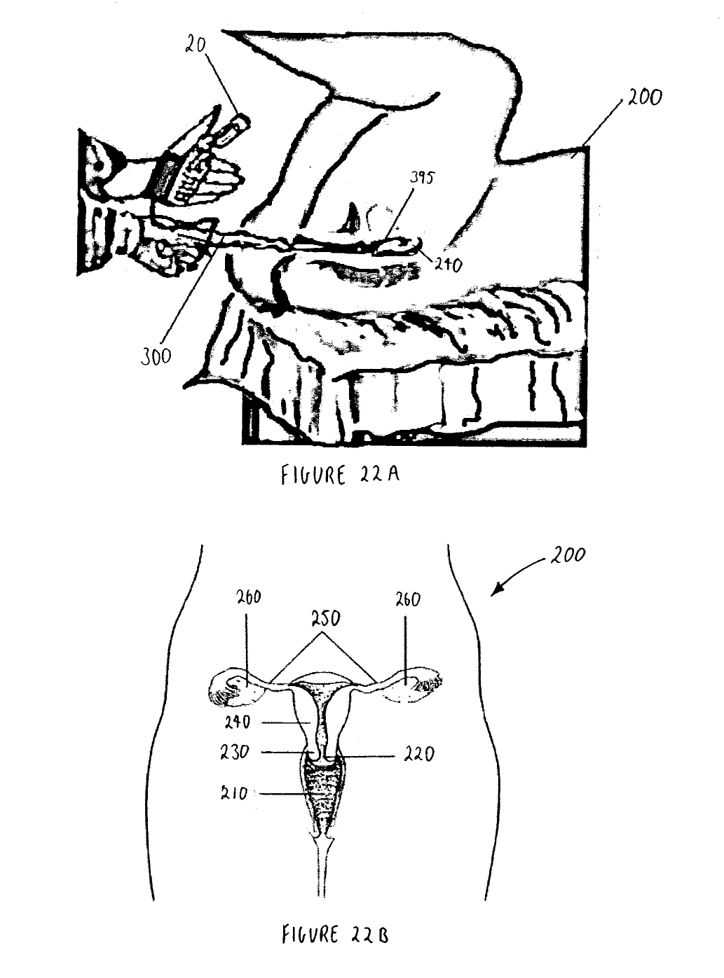

The endoscope (300) having a distal portion covered by the balloon sheath (390) may be used for imaging the female reproductive system (200) as shown in FIG. 22. The distal end (395) of the endoscope (300) is inserted into the female vagina (210) by a doctor or nurse. The endoscope (300) is manipulated to enter the cervix (230) through the cervical canal (220), the procedure being aided by the flexible nature of the endoscope (300). The distal end (395) enters the uterus or womb (240) and the doctor or nurse inflates the balloon (310) by depressing the plunger (322) of the syringe (320), as hereinbefore described in more detail. The inflated balloon (310) opens the wall of the cervix (230) and maintains the distal end (395) of the endoscope (300) in place in the uterus (240). Light is emitted from the distal end (395) of the endoscope (300) and illuminates the uterus (240) and the opening of the fallopian tube (250). An image of the fallopian tubes (250), which are connected to the ovaries (260), is transmitted to the image-viewing device (20). The doctor or nurse can instantly view the image on the display (LCD) screen (21).

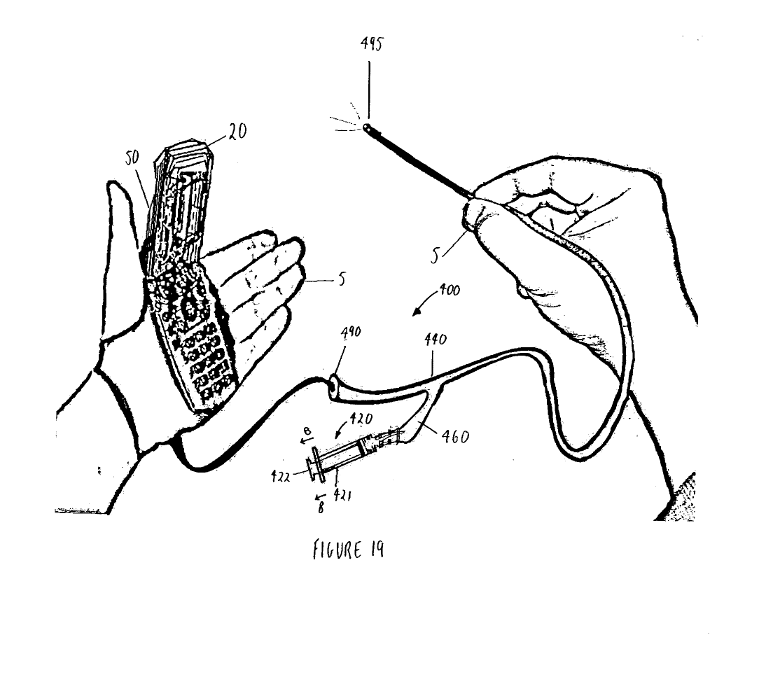

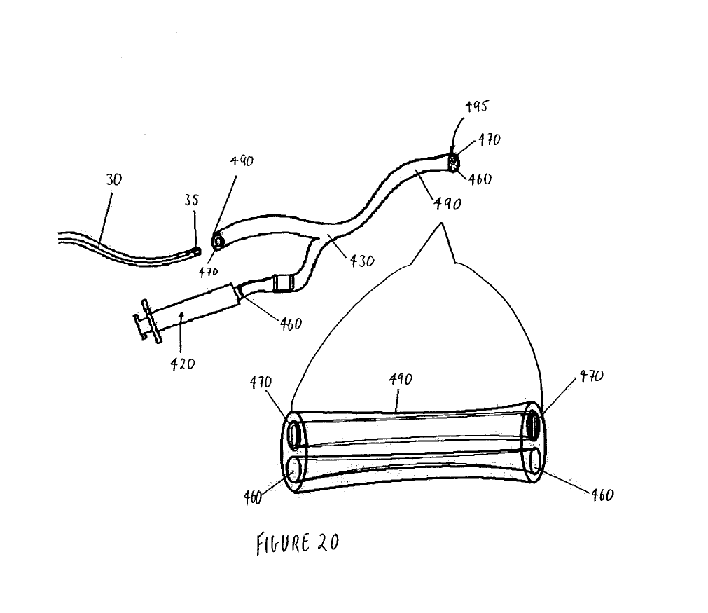

In another preferred embodiment of the present invention, the sheath of the portable imaging apparatus includes a suction conduit with an open distal end at or adjacent the distal end of the sheath and a proximal end connected to suction means. FIGS. 19 and 20 show an example of an imaging apparatus of the present invention with the endoscope (400) having a suction sheath (490) comprising a suction tube (460) connected to a syringe (420) with a body (421) and a plunger (422). The end of the body (421) of the syringe (420) is connected to suction tube (460) so that when the plunger (422) is pulled out in direction B, a suction force is created in suction tube (460). The suction force may be used to collect cellular sample or other samples from a body cavity or passage into which the endoscope (400) is inserted, for example for use in taking a biopsy from the fallopian tubes (250) shown in FIG. 22B. As well as collection of samples, the endoscope (400) can be simultaneously used to capture an image of the fallopian tubes (250). This embodiment of the apparatus of the present invention may be used for suction of wound cavities or the like. The apparatus provides an image of the wound cavity so that targeted suction can be performed.

Suction sheath (490) has an endoscope tube (470) for receiving the endoscope (30). The distal end (35) of the endoscope (30) is inserted into endoscope tube (470) until it reaches the closed distal end of the sheath (495), so that the sheath (490) surrounds the distal portion of the endoscope (30). At junction (430) of the sheath (490), suction tube (460) meets endoscope tube (470), which in use contains endoscope (30). The sheath (490) is preferably substantially liquid and gas impermeable and acts as a barrier to protect the endoscope (30) and in particular the lens (31) at the distal end (35) of the endoscope (30).

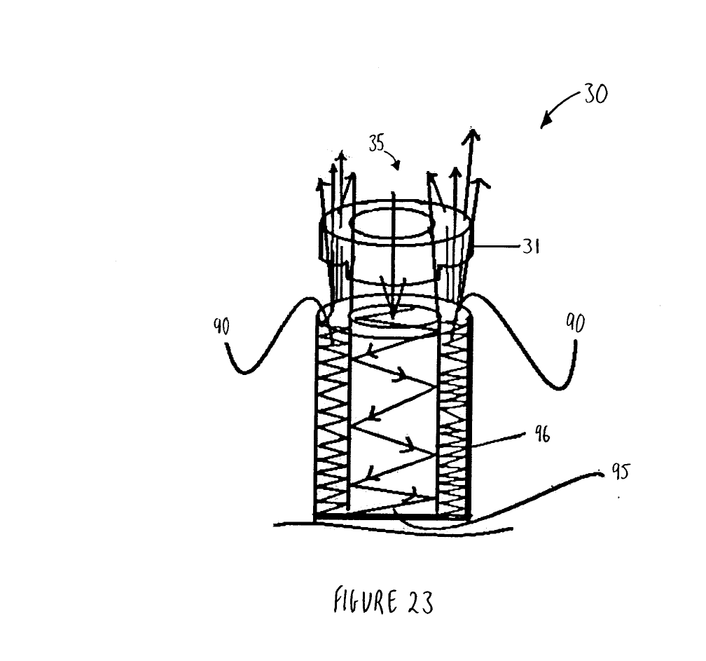

In a further preferred embodiment of the imaging apparatus of the present invention, the sheath includes a grasper at the distal end thereof for grasping an object. FIG. 24 shows an example of an imaging apparatus of the present invention with the endoscope (500) having a grasper sheath (590) comprising a grasper tube (560) connected to a syringe (520) with a body (521) and a plunger (522). The grasper tube (560) is provided with a grasper comprising at its distal end grasper claws (510) and a connector that connects the grasper claws (510) to the syringe plunger (522). The syringe body (521) is connected to grasper tube (560) and the syringe plunger (522) is used to manipulate the grasper claws (510) at the distal end (595) of the endoscope (500). The grasper claws (510) may be used to grasp an object, such as a foreign body or tissue sample from a body cavity or passage into which the endoscope (500) is inserted. As well as grasping objects, the endoscope (500) can be simultaneously used to capture an image of the internal body area.

Grasper sheath (590) has an endoscope tube (570) for receiving the endoscope (30). The distal end (35) of the endoscope (30) is inserted into endoscope tube (570) until it reaches the closed distal end of the sheath (595), so that the sheath (590) surrounds the distal portion of the endoscope (30). At junction (530) of the sheath (590), grasper tube (560) meets endoscope tube (570), which in use contains endoscope (30). The sheath (590) is preferably substantially liquid and gas impermeable and acts as a barrier to protect the endoscope (30) and in particular the lens (31) at the distal end (35) of the endoscope (30).

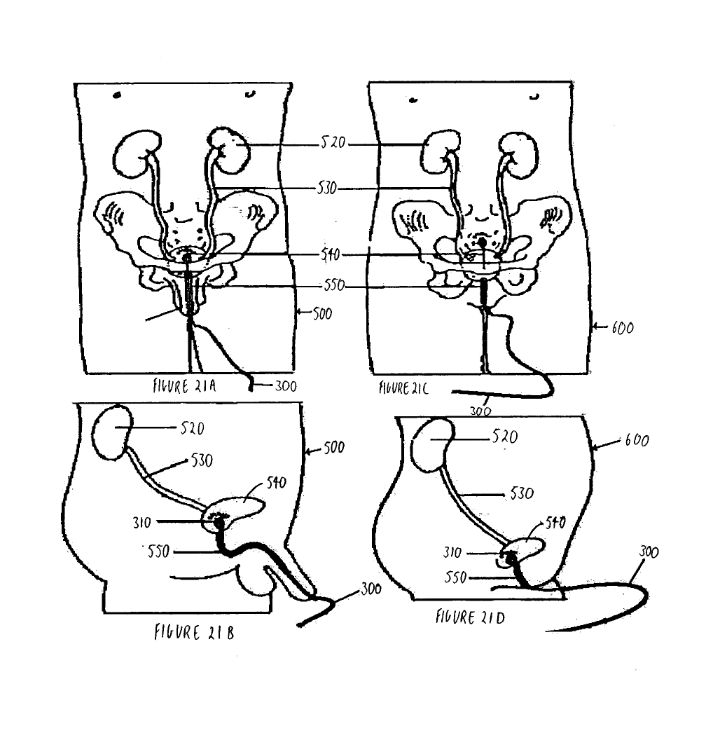

FIG. 21 shows examples of different uses of the apparatus of the present invention. In FIG. 21, the endoscope (300) includes a balloon sheath (390) as hereinbefore described in connection with FIGS. 14 to 18, however, other examples of endoscopes such as the endoscope (400) with the suction sheath (490) or the endoscope with the protective sheath as hereinbefore described in more detail could be used instead.

FIG. 21 shows the male (500) and female (600) urinary system comprising two kidneys (520) connected to a bladder (540) by two ureters (530). Urine passes from the bladder (540) out of the body via the urethra (550). To view images of the internal spaces of the urinary system, the distal end (395) of the flexible endoscope is inserted into the urethra (550) of the male (500) or female (600) by a doctor or nurse and manipulated to pass into the bladder (540). Once in the bladder, the balloon (310) is inflated to maintain the distal end (395) in position. Light is emitted from the distal end (395) of the endoscope (300) to illuminate the internal space of the bladder (540) and an image is transmitted back to the Charge-Coupled Device (CCD) image sensor (60) of the image-viewing device (20). The image can be viewed by the doctor or nurse on the LCD screen (21) of the image-viewing device (20). The image can also be stored or further manipulated by the image viewing device, for example the image may be magnified to focus in on a particular area of interest. The image can also be transferred to a computer or the like typically via a USB port in the image-viewing device (20). The balloon (310) may also function as a speculum, allowing the doctor or nurse to take a biopsy sample of the bladder.

The portable imaging apparatus of the present invention can be used to view an image of any internal area, which cannot be view from the outside, such as internal body cavities or organs of humans or animals. Provided the endoscope can be inserted into the object, the image-viewing device can display images of the internal spaces of that object. There is no need to transfer the image to an external image display device such as a computer. The image-viewing device can be held in a human palm and the imaging apparatus of the present invention is therefore easy to use and portable. The endoscope may be inserted into any body channel or canal, such as the urinary track, ear, nose, oesophagus, anus or reproductive system, or into an opening made by surgical intervention or a wound. Images of internal body cavities, organs or passageways can thus be viewed. Instant visualisation of an image often allows detection of pathologies that have passed unnoticed by previous diagnostic procedures. Once the diagnosis has been made, endoscopic surgery may be carried out. The advantages of endoscopic surgery are many, including smaller incision and thus less scaring, milder post-operative pain, and shorter hospitalization. In addition the apparatus of the present invention may be used to collect biopsy samples from within internal body cavities, organs or passageways.

The portable imaging apparatus of the present invention is particularly suitable for use in the field of urology for imaging and diagnosing infections in the urinary track, and in obstetrical and gynaecological practices in particular for use in the early diagnosis of ovary cancer or the like.

Use of the imaging apparatus of the present invention is not limited to medical or veterinary practices, the apparatus may be used in any situation where an endoscope is used to access and transmit images of internal areas, which cannot be adequately viewed from the outside. For example the apparatus of the present invention may be used for inspection of internal parts of machines or the like.

The present invention has been described with regard to preferred embodiments. However, it will be obvious to persons skilled in the art that a number of variations and modifications can be made without departing from the scope of the invention as described herein.

1. A portable imaging apparatus comprising:
an endoscope comprising an image capture optical pathway and an illumination optical pathway; and
a hand holdable image-viewing device releasably connectable to a proximal end of the endoscope, the device comprising:
(i) an image detector optically couplable to the image capture optical pathway, for detecting an image transmitted from a distal end of the endoscope;
(ii) a display in communication with the detector, for displaying an image detected by the detector; and
(iii) a light source optically couplable to the illumination optical pathway, for transmitting light to the distal end of the endoscope.

2. The imaging apparatus of claim 1, wherein the image-viewing device includes a power source.

3. The imaging apparatus of claim 2, wherein the power source is rechargeable.

4. The imaging apparatus of claim 1, wherein the image capture optical pathway comprises a plurality of image capture fibre optics and the and illumination optical pathway comprises a plurality of illumination fibre optics.

5. The imaging apparatus of claim 4, wherein the illumination fibre optics surrounds the image capture fibre optics along at least a length of the endoscope.

6. The imaging apparatus of claim 1, wherein the proximal end of the endoscope comprises an adaptor releasably connectable to the image viewing device, wherein the adaptor preferably comprises a first connector in optical communication with the illumination optical pathway and a second connector in optical communication with the image capture optical pathway.

7. The imaging apparatus of claim 1, wherein the image-viewing device includes a port releasably connected to the proximal end of the endoscope, the port comprising an image connector optically couplable with the image capture pathway of the endoscope and a illumination connector optically couplable with the illumination pathway of the endoscope.

8. The imaging apparatus of claim 1, wherein the light source comprises a light booster system comprising a Light Emitting Diode (LED) in communication with a lens and a programmable chip.

9. The imaging apparatus of claim 1, including a removable sheath configured to cover a distal portion of the endoscope, the sheath comprising an endoscope conduit with a closed distal end and an open proximal end for receiving the distal end of the endoscope, the sheath comprising a substantially liquid impermeable material.

10. The imaging apparatus of claim 9, wherein the sheath comprises a substantially gas and liquid impermeable material.

11. The imaging apparatus of claim 9, wherein the sheath comprises substantially transparent latex material.

12. The imaging apparatus of claim 9, wherein the sheath includes an inflatable balloon approximate the distal end of the sheath.

13. The imaging apparatus of claim 12, wherein the balloon is connected to an inflator by an inflation conduit, wherein the balloon is inflated by fluid which passes from the inflator to the balloon via the inflation conduit.

14. The imaging apparatus of claim 13, wherein the inflator comprises a syringe connected to the inflation conduit.

15. The imaging apparatus of claim 9, wherein the sheath includes a suction conduit with an open distal end at or adjacent the distal end of the sheath and a proximal end connected to suction means.

16. The imaging apparatus of claim 15, wherein the suction means comprises a syringe.

17. The imaging apparatus of claim 9, wherein the sheath includes a grasper at the distal end thereof for grasping an object.

18. The imaging apparatus of claim 1, wherein the image-viewing device comprises a mobile phone.

19. The imaging apparatus of claim 1, wherein the image-viewing device comprises a Personal Digital Assistant (PDA).

20. A hand holdable image-viewing device comprising:
(i) a port releasably connected to a proximal end of an endoscope, the port comprising an image connector optically couplable with an image capture pathway of the endoscope and an illumination connector optically couplable with an illumination pathway of the endoscope
(ii) an image detector optically coupled to the image connector;
(iii) a display in communication with the detector; and
(iv) a light source optically coupled to the illumination connector.

21. The image-viewing device of claim 20 further including a power source.

22. The image-viewing device of claim 21, wherein the power source is rechargeable.

23. The image-viewing device of claim 20, further including a controller.

24. The image-viewing device of claim 20, wherein the light source comprises a light booster system comprising a Light Emitting Diode (LED) in communication with a lens and a programmable chip.

25-28. (canceled)
